(19) United States
(12) Patent Application Publication
(10) Pub. No.: US 2010/0234794 A1
(43) Pub. Date: Sep. 16, 2010

(54) SYSTEM AND METHOD FOR REDUCING SURGICAL SITE INFECTION

(76) Inventors: Kevin Shaun Weadock, Hillsborough, NJ (US); Robert Nering, Stockton, NJ (US)

Correspondence Address: PHILIP S. JOHNSON;JOHNSON & JOHNSON, ONE JOHNSON & JOHNSON PLAZA, NEW BRUNSWICK, NJ, 08933-7003, US

(21) Appl. No.: 12/402,631

(22) Filed: Mar. 12, 2009

(51) Int. Cl. A61M 37/00; A61N 5/06

(52) U.S. Cl. 604/20; 604/23; 604/24

(57) ABSTRACT

A system and method for performing a surgical procedure that are based on delivering air to a surgical site to prevent contamination of a wound or surgical instrument. A curtain of air above the surgical site prevents pathogens or pathogen laden particulates eminating from sources such as the skin of health care professionals including operating room staff, room air, and ceiling from contaminating the surgical site. The curtain of air may be turbulent or laminar and heated or humidified with a solution of anti-microbial agent such as an antibiotic or anti-microbial such as triclosan. Optional incorporation of a UV or blue light source into the system may also prevent infection. The system is designed to be ergonomically compatible with existing surgical substrates such as a retractor, bed, or drape. Sources of sterile air can be brought to a manifold pivotably coupled to a shaft that is attached to a bedrail or instrument stand to deliver the air directly over a surgical site.

### FIELD OF THE INVENTION

The present invention generally relates to a system and method for reducing the incidence of surgical site infection, and more specifically to apparatuses and methods for using and directing gases useful in reducing the incidence of surgical site infection.

### BACKGROUND OF THE INVENTION

Infections contracted in hospitals and other health care facilities are the fourth largest killer in America. Every year in this country, almost two million patients contract infections in hospitals, and an estimated 103,000 die as a result, as many deaths as from AIDS, breast cancer, and auto accidents combined. These deaths are largely due to respiratory system infections, urinary tract infections, catheter related infections, and surgical site infections resulting from accidental exposure to pathogens. The risk of surgical site infection is present in all surgical procedures, but can be particularly serious in certain operations, such as cardiovascular surgery and joint replacement.

Rigorous adherence to the principles of asepsis is the foundation of surgical site infection prevention. It is critical to minimize a patient's exposure to bacterial contamination (as well as other pathogens) from members of the surgical team and from non-sterile equipment or surfaces in the operating room through the use of gloves, gowns, masks, and drapes. Draping of the surgical site provides a sterile work surface and helps minimize the transfer of microorganisms between non-sterile areas and the surgical wound. These measures also help protect health care professionals from exposure to pathogens in the patient's blood and other body fluids.

It has been long recognized that hand hygiene is also a very important exercise to reduce infection in the operating room and in other patient locations within a health care facility. Surgeons and staff are trained to wash their hands extensively prior to putting on sterile gloves. In addition, solutions with anti-microbial activity are frequently used to scrub the patient and prospective surgical site prior to surgery. Surgeons and all personnel within the sterile field wear sterile gowns, gloves, and masks. Their hair is typically covered with a hair net or hat. Prophylactic antibiotics may also be given to the patient as part of the surgical preparation. In addition, all instruments and medical devices are sterilized prior to the procedure.

Despite compliance of the surgeon and surgical staff with rigorous hygiene principals and infection control protocols, airflow in the operating room can affect infection rates by allowing certain bacteria to get to a wound or other access site in a patient. The bacteria can be blown into the wound from health care providers' (surgeon, nurses, anesthesiologist, technicians, etc.) skin, hair, clothing, or hands. In addition, bacteria or other pathogens can contaminate an open wound, for example, when entrained in air entering the operating room while a door to the operating room is open. Bacteria or other pathogens from a prior surgical procedure or cleaning exercise by cleaning staff may become airborne. As a result, and in an attempt to mitigate and/or prevent such contamination, laminar flow of HEPA-filtered air has also been employed in the operating room to reduce scatter of bacteria into a surgical wound. Conventional laminar flow systems operate by drawing ambient air, under negative pressure, into a laminar flow unit. This air first passes through a pre-filter which traps the larger size dust and dirt particles. A blower in the unit then directs this pre-filtered air, now under positive pressure, through a conventionally-known 99.97% efficient HEPA filter to generate sterile, unidirectional ultra-clean air. The HEPA filter can remove particles down to a size of 0.30 microns. Viruses range in size from 0.01 microns to 0.03 microns.

Bacteria range in size from 0.1-15 microns. Other pathogens such as protozoa can be even larger. It is also recognized that many pathogens are typically attached to dust particles, or contained in droplets, although they may be also individually entrained in an air stream or draft. When the laminar air moves in one direction at a uniform speed of between 70-120 FPM, its individual molecules assume parallel paths, or streamlines. The physics of this phenomenon allow for these streamlines of air to bend around objects and obstacles without losing laminarity or losing the particles that they carry. Currently, the use of laminar flow air involves directing HEPA filtered air from wall or ceiling vents to floor vents. Because of the parallel stream lines, entrained particles will remain entrained in the airstream until a turbulent condition is encountered.

Such conventional ventilation systems are widely used in operating rooms in many countries around the world. These systems entail high investment costs and operating expenses, and must be properly and constantly maintained to be effective. A recent study by Brandt, et al. (Operating Room Ventilation with Laminar Air Flow Shows No Protective Effect on the Surgical Site Infection Rate in Orthopedic and Abdominal Surgery; Annals of Surgery, Volume 28, Number 5, Pages 695-700, November, 2008) evaluated whether operating room ventilation with vertical laminar airflow impacts surgical site infection rates. Surprisingly, they found that operating room ventilation with laminar airflow showed no benefit and was, surprisingly, even associated with a significantly higher risk for severe infections. The authors hypothesized that the reason for this surprising finding is that the heads of the surgical team members may be positioned above the surgical site, i.e., directly in the laminar airstream from the ceiling down to the wound. This may facilitate pathogen containing particles such as droplets and skin particles, falling directly into the wound with the downstream airflow. Another hypothesis proposed by the authors was that the ventilation may allow cooler air to fall into the wound, lowering intra-operative tissue temperatures. Lowering of body temperature is known to increase the chance of surgical site infection, and a local decrease in temperature could theoretically increase the chances of surgical site infection as well.

Unfortunately, current ventilation systems designed to reduce surgical site infections are expensive to install and maintain and may still allow bacteria to enter the surgical wound from any non-sterile, shedding surfaces that are very close to the surgical wound. This is evident since surgical site infection remains a source of illness and possible cause of death in the surgical patient. In addition, infections acquired during surgery invariably result in longer hospitalization and higher costs. Hospitals may soon be denied reimbursement costs for those cases where an infection was deemed to be acquired in the hospital, i.e., a so called “never event”. Accordingly, there is a need in this art for novel apparatuses and methods for delivering gases directly over or in close proximity to a wound or surgical site in order to reduce or prevent pathogens from contacting or entering the wound, and thereby reduce the incidence of infections.

### SUMMARY OF THE INVENTION

It is an object of the present invention to provide a system for preventing or reducing the incidence of infection during a surgical procedure or during treatment in a healthcare facility. Therefore, a system is disclosed for preventing infection of a surgical site, wherein the surgical site is a wound or instrument. The system is comprised of a source of air that is coupled to a manifold having a wall, a proximal end, distal end, and a lumen therein. The air enters the lumen of the manifold and then exits at least one opening in the wall of the manifold. The manifold is attached to a substrate proximate a surgical site by an attachment means or attachment member such as clamp, a shaft, bracket, screw, adhesive, cable, chain, wire, staple, or velcro. The substrate is typically a surgical retractor, bed, bedrail, or instrument stand. Preferably, the attachment means or attachment member is a shaft having a proximal end and a distal end, the proximal end adapted to be secured to the substrate, and the distal end of the shaft attached to the manifold. Air exiting the manifold passes directly over the surgical site so as to prevent airborne pathogens such as bacteria and pathogen- laden particles from entering or contaminating the surgical site.

In one embodiment, the source of air is comprised of a power supply, a a fan or blower, a filter, and coupling means for delivering the air to the manifold. In another embodiment, the source of air can be a compressed air source, e.g., in a tank under pressure. The air may contain carbon dioxide or a dilute solution of anti-microbial agent or antibiotics. In another embodiment, the shaft is flexible or articulated and is pivotably coupled to the manifold. In yet another embodiment, the system further comprises an ultraviolet or blue light source producing a wavelength within the range of 200-400 nm or 440-490 nm, respectively. Antibiotic-resistant bacterial infections represent an important and increasing public health threat. At present, fewer than 5% of staphylococcal strains are susceptible to penicillin, while approximately 40%-50% of Staphylococcus aureus isolated have developed resistance to newer semisynthetic antibiotics such as methicillin. In still yet another embodiment, the light emits blue light with a wavelength of 440-490 nm, preferably at 470 nm. This light has been demonstrated to be effective in killing methicillin resistant Staphyloccoccus aureus (MRSA).

Another aspect of the present invention is a method for performing a surgical procedure, the method includes providing a source of air that is coupled to a manifold having a wall, a proximal end, distal end, and a lumen therein. The air enters the lumen of the manifold and then exits at least one opening in the wall of the manifold. The manifold is attached proximate a surgical site by attachment means including a clamp, a shaft, bracket, screw, adhesive, cable, chain, wire, staple, or velcro. The substrate is typically a surgical retractor, bed, bedrail, or instrument stand. The substrate as contemplated by the present invention can also be a surface of the patient's body. Preferably, the attachment means useful in the method of the present invention is a shaft having a proximal end and a distal end, the proximal end adapted to be secured to the substrate, and the distal end of the shaft attached to the manifold. Air exiting the manifold passes directly over the surgical site so as to prevent airborne bacteria and bacteria laden particles from entering the surgical site.

In one embodiment, the source of air is comprised of a power supply, a fan, a filter, and coupling means for delivering the air to the manifold. In another embodiment, the method mixes the source of air with carbon dioxide, an anti-microbial agent, or an antibiotic to further aid in preventing contamination of the surgical site.

In yet another embodiment, the method includes adding an ultraviolet light (200-400 nm) or blue light (440-490 nm) source to the system.

In still yet another embodiment, the method includes providing a plurality of these devices, with at least one device coupled to a negative source of air and at least one device coupled to a positive source of air.

These and other aspects and advantages of the present invention will become more apparent from the following description and examples, and accompanying drawings.

### BRIEF DESCRIPTION OF THE DRAWINGS

FIGS. 1A and 1B illustrate an embodiment of a system of the present invention having a shaft coupled to a surgical retractor; the distal end of the shaft is seen to be coupled to a non-cylindrical manifold that distributes a curtain of air over a surgical site;

FIG. 2 illustrates another embodiment of a system of the present invention having an articulating shaft coupled to a surgical retractor; the distal end of the shaft is seen to be coupled to a cylindrical manifold that distributes a curtain of air over a surgical site;

FIG. 3 illustrates another embodiment of a system of the present invention illustrating a standard surgical retractor that has been modified to provide a curtain of filtered air over a surgical site;

FIG. 4 illustrates another embodiment of the present invention having an opposed pair of flexible manifolds that are coupled to a source of air and have an adhesive backing to enable mounting to a variety of surgical substrates, including the patient's skin.

FIG. 5 illustrates another embodiment of the system of the present invention having a bunker style manifold to distribute a curtain of air over a surgical site.

FIGS. 6A and 6B illustrate another embodiment of the system of the present invention illustrating a device that is suspended from a ceiling and provides at least one layer of laminar flow air over the surgical site.

FIG. 7. illustrates a schematic of a system of the present invention having the optional capability of using variations of air, UV light, blue light, CO₂, and nebulized anti-microbial agents to reduce the likelihood of surgical site infection.

### DETAILED DESCRIPTION OF THE INVENTION

Although the disclosure hereof is detailed and exact to enable those skilled in the art to practice the invention, the physical embodiments herein disclosed merely exemplify the invention that may be embodied in other specific structure and are not limited thereto. While the preferred embodiments have been described, the details may be changed without departing from the spirit and scope of the invention, which is defined by the claims.

As used herein the term “laminar flow” is defined to mean a flow of air moving with uniform velocity and direction with a minimum amount of turbulence. The term “turbulent flow” is defined to mean a flow of air having a non-uniform or random direction with varying velocity. The term “air stream” is defined to mean air exiting a manifold and having a general direction with varying degrees of laminar and turbulent properties. The term “surgical site” is defined to mean a surgical wound, a traumatic wound, or a table or instrument required for surgery

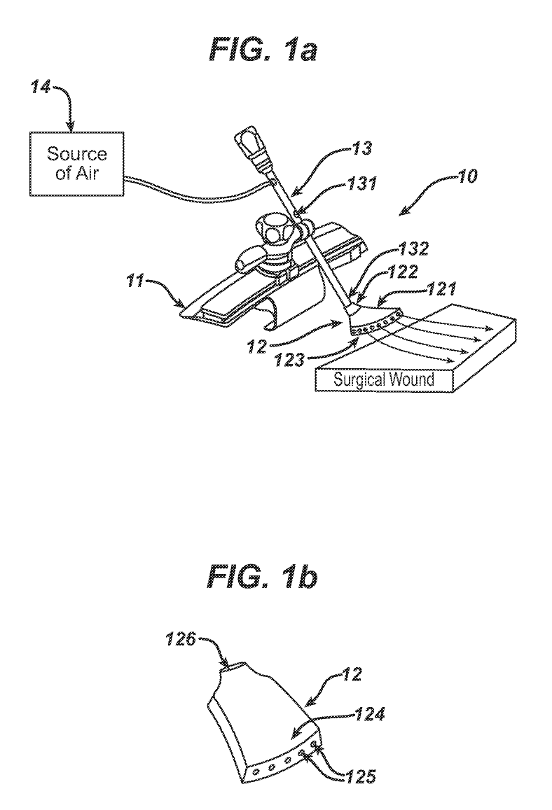

FIGS. 1A and 1B illustrate one embodiment of the system of the present invention that is mounted onto a standard retractor used for abdominal, thoracic surgery, or orthopedic surgery. The system 10 is mounted to the retractor 11 so that the manifold 12 can deliver filtered air over the surgical site. The system 10 is seen to have a shaft 13 having a proximal end 131 and a distal end 132. A manifold 12 is coupled to the distal end 131 of the shaft 132. The manifold 12 has wall 121, proximal end 122, distal end 123, and at least one lumen 124 therebetween. The wall 121 is also seen to have a top, sides, and a bottom. The lumen 124 is seen to be coupled to a source of air 14. There is at least one opening 125 in the wall of the manifold 12 in communication with the lumen 124 to allow an air stream 140 to exit and flow over surgical site 150. As illustrated in FIG. 1A, the proximal end 131 of the shaft 13 can be coupled or mounted to a conventional surgical retractor 11. In other embodiments, the proximal end 131 of the shaft 13 can be coupled to other structures such as a railing on a surgical bed, a surgical instrument stand, or any other suitable piece of equipment found in a typical operating room or health care provider setting. The shaft 13 can optionally be a flexible shaft formed of articulating segments that are simply locked together by a cable running through the segments. The shaft 13 can be made from conventional biocompatible materials used to construct medical devices including polymers or metal such as stainless steel or nitinol. A nitinol shaft has the advantage of enabling the device to be deflected easily during the procedure, if necessary in order to redirect the airstream. The shaft 13 may have any length and diameter (cross-section) sufficiently effective to support a manifold 12 and permit adjustment of the airstream with respect to the surgical site. Preferably, the length of the shaft 13 is within a range of about 1-30 cm, and the diameter of the shaft 13 is in a range of about 1-15 mm. It will be appreciated that the cross-section of the shaft may have a variety of configurations, including circular, elliptical, square, rectangular, etc. The distal end 132 of the shaft 13 can be coupled to the center 121 of the manifold 12 or to one end, for example, the proximal end 122 of the manifold 12. In one embodiment, sterile air under positive pressure is delivered from a fan or blower.

The manifold 12 illustrated in FIG. 1B is a housing with at least one side having an opening 126 or other means to be coupled to the source of air. Suitable constructs for coupling the manifold to the source of air include a flange, a lock-release mechanism used in the compressed gas industry, or other means known to those skilled in the art of coupling mechanisms with fluid or air mediums. Conventional tubing, piping or ducts and the like made of metal or polymer can also be used to couple the source of air to the manifold. The shape of the manifold 12 can be square, circular, cylindrical, elliptical, egg shaped, rectangular, triangular, or any other suitable shape. Multiple lumens are illustrated exiting holes 125 on one side of the manifold 12. The air entering the manifold 12 can be diverted into a number of independent air streams by having a plurality of lumens 124 within the manifold. This will result in an air stream that is more laminar in profile. Alternatively, the manifold can be a single lumen or chamber that distributes air to at least one hole 125 on the same side of the manifold 12 or any one or more sides of the manifold, resulting in an air stream that is more turbulent in profile. The manifold 12 can be made from conventional materials used to construct medical devices including plastic, metal or ceramic or any combination thereof. The manifold 12 can be a singular structure that is rigid, flexible, or bendable. Alternatively, the manifold 12 can be fabricated from at least two units linked together to provide for articulation. This articulation can allow for added configurations of the manifold.

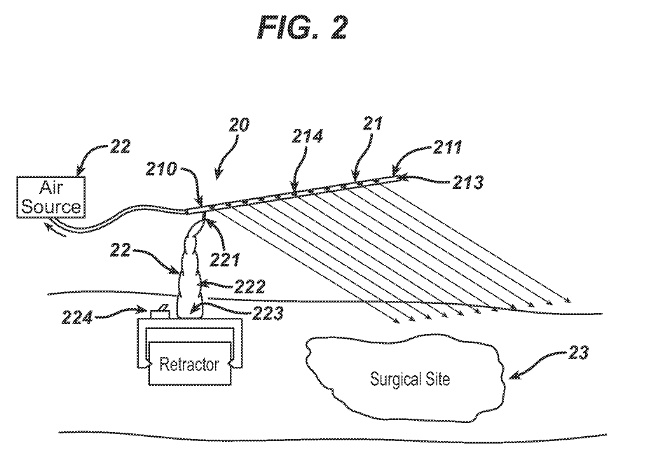

FIG. 2 illustrates one embodiment of a system 20 of the present invention wherein the manifold 21 is a cylindrical or tubular member having a side wall 215, a proximal end 210 and a distal end 211 and a lumen 213 therein. Proximal end 210 is seen to have an opening in communication with lumen 223. In the embodiment illustrated in FIG. 2, the proximal end 210 of the manifold 21 and lumen 223 therein are coupled to the source of air 22. The length of the cylindrical manifold 21 preferably ranges from about 5 cm to 60 cm, depending on the size of the patient and the surgical procedure to be performed, although any sufficiently effective and suitable size may be used. The diameter of the manifold is sufficiently effective to provide desired air flow and can preferably range from about 2 mm -25 mm. A varying diameter or cross-section of the manifold or the lumen within the manifold may also be used. This allows the velocity of air flow to be controlled, i.e., velocity of air flow can be increased at a desired point on the manifold by decreasing the lumenal diameter or cross-section within the manifold. The manifold can have a variety of cross-sectional profiles, including but not limited to circular, triangular, flat, rectangular, elliptical, square, etc.

Various shapes for the air holes 214 can be formed so as to control the direction, velocity, and turbulence within the air flow. For example, the air holes can be rectangular, triangular, circular, or elliptically shaped. The air holes 214 extend through side wall 215 and are in communication with lumen 223. If desired, two manifolds of the present invention can be placed adjacent to one another, with one manifold delivering air at a first velocity and another manifold delivering air at a second velocity. Air exiting holes 214 in the manifold 21 is directed immediately over a surgical site, thereby reducing the likelihood of foreign material such as airborne bacteria, epithelial cells, dust, or bacteria laden microparticles from entering the site. The air delivered can be in a laminar form or non-laminar (turbulent) curtain of air. The air curtain can be delivered parallel to the surgical site and the height of the air curtain relative to the surgical site is easily adjusted by adjusting the shaft 22 or the coupling point 221 between the shaft 22 and the manifold 21. For a shaft having articulating members 222 disposed over a cable 223, the height of the manifold relative to the surgical site can be adjusted by loosening the cable 223 within the shaft 22, adjusting the height, and then applying tension to the cable 223 by using a tensioning arm 224 or equivalent. Other means for adjusting the height can be to use magnetorheological or electrorheological fluids within the shaft 22. In the embodiment that uses magnetorheological or electrorheological media in the shaft, a magnetic or electric field would also need to be supplied to the shaft 22, respectively. Higher heights of the air stream relative to the surgical site allow for the surgeon's hands and tools to operate below the airflow stream. Lower heights reduce the chance bacteria can enter the site, but are somewhat compromised by turbulence created by the surgeons hands and instruments. Nonetheless, the air stream can reduce airborne bacteria and particulates such as skin cells, hair, dust, etc. from entering the surgical site. In the preferred embodiments of the system and method, the air is delivered proximate a surgical site, preferably within about 2-36 inches above the sitedepending upon the operating room environment, the surgical procedure being performed, and other circumstances. Delivering the air proximate a surgical site, with no potential sources of contamination such as people or non-airborne bacteria between the surgical site and the air leaving the manifold, therefore reduces or eliminates the likelihood of contamination of the surgical site. The direction of airflow can be adjusted by tilting the manifold in one or more directions. Thus, airflow can move in a vertical direction if an air wall is needed or desired. Alternatively, the manifold 21 delivers air in a direction perpendicular to the surgical site 23. The air stream can be adjusted to be laminar, laminar-like or turbulent when using the systems of the present invention. For a more laminar air flow, a straight manifold having a fixed lumenal diameter of about 1-5 mm can be used. For a more turbulent air flow, larger lumens with varying diameters of about 6-10 mm can be employed. The lumenal surface of the manifold can also be roughened to enhance turbulence. The manifold can also be curved to increase turbulence

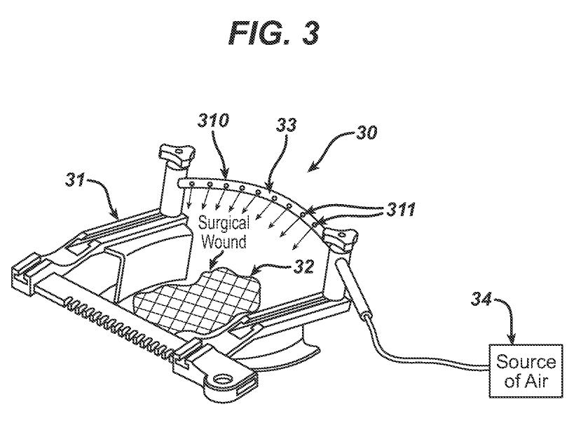

Referring now to FIG. 3, a system 30 incorporated into a surgical retractor 31 is illustrated. In this embodiment, the surgical retractor 31 has been modified so that it can deliver air directly over a surgical site such as a wound 32. A hlumen 33 exists within the arm 310 of the retractor 31. Positive pressure air flow is supplied by the source of air 34 and exits as a plurality of air streams 312 through a plurality of ports 311 along the length of the arm 310. The angle of the air exiting the arm can be adjusted by rotating the arm 310 slightly. Thus, air in the form of air streams 312 is delivered proximate a surgical site such as a surgical wound 32.

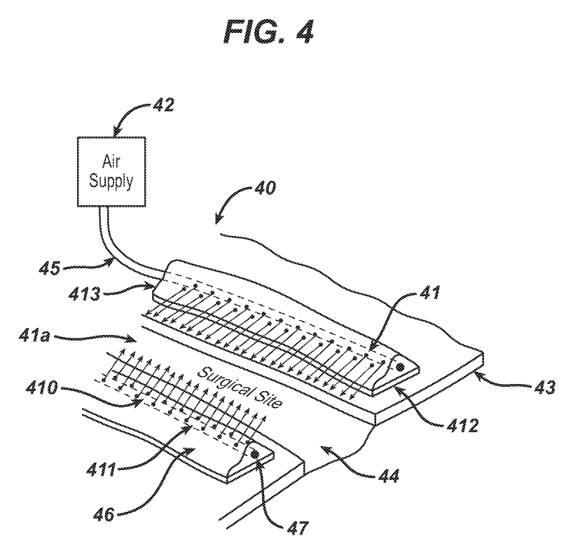

FIG. 4 illustrates one embodiment of a system 40 of the present invention having a manifold 41 coupled to a source of air 42 by a plastic or metallic tubing or other conventional conduit 45. As illustrated, two manifolds 41 and 41a are disposed around a surgical site 44 by attaching the manifolds to a substrate 43. Each manifold has an interior lumen 48. Typical substrates are selected from the group consisting of a surgical drape, a bed railing, a retractor, or other substrate near the surgical site, and even the patient's skin at the option of the surgeon. The source of air 42 is coupled to the proximal end 413 of the flexible manifolds 41 and 41a. In one embodiment, the source of air is coupled to the central region 46 of the manifolds 41 and 41a. The end or ends of the manifolds 41 and 41a that are not coupled to the air source 42 can be plugged with a plug 47 or pinched or sealed off to avoid pressure losses. Alternatively, one end of the manifold can receive air alone and the other end can be coupled to a source of humidified air or carbon dioxide or nebulized solution of anti-microbial or antibiotic agent. The manifolds can be placed on existing retractors or any surface near the surgical site. They preferably have at least one surface to which an adhesive layer or strip 412 is applied. The strip is covered with a protective peel-off layer that is removed just prior to applying to the desired surface. In one embodiment, the manifold is flexible, with at least one side having an adhesive strip disposed thereon. The manifolds can be comprised on known biocompatible materials such as polypropylene, polycarbonate, silicone, polyurethane, polyethylene, silicone, or Teflon. The holes or openings 411 in the manifolds 41 and 41a are in communication with lumen 48 and are preferably made with a laser but may also be made in any conventional manner such as by puncturing the manifold at regular spatial intervals with a punch and die, drilling, etc., for example, every cm. The manifolds 41 will have a sufficiently effective number of holes 411, preferably about 1-20 holes or openings 411 on the side where air 49 is exiting. The holes 411 can be any shape but are preferably circular. The diameter or cross-section of the holes is sufficient to effectively provide the desired air flow, for example, about 0.1-1 mm.

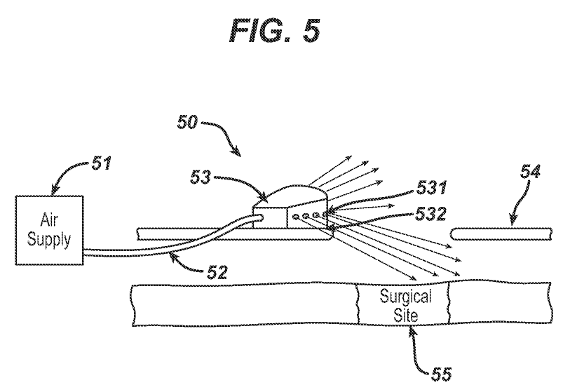

FIG. 5 illustrates yet another embodiment 50 of the present invention having a “bunker-like” manifold 53 that has at least one side with an adhesive 532. The bunker manifold 53 can be placed at discrete locations during the surgical procedure. A plurality of the manifolds 53 can be placed near the surgical site 55. The bunker manifold 53 is seen to have member 51 with interior lumen or cavity 57. This embodiment provides a great deal of freedom to the surgical staff with respect to exactly where and how each bunker manifold 53 delivers air. HEPA filtered air 58 can be supplied from an air source 51 and through a tube 52 to the interior 57 of bunker 53. Once coupled to the bunker manifold 53, the air 58 can be directed through openings 531 (in communication with lumen 57) in any number of directions and on multiple surgical sites, if present. In addition to an adhesive backing 532 as a way of attaching to substrates 54 such as drapes, retractors, rails, clothing, etc., other conventional devices or components can be used such as velcro, pins, staples, tape, snaps, buttons, or sutures and the like. The bunker manifolds 53 can have a sufficiently effective number of openings 531, for example about 1-20 openings 531, on the side where air is exiting. As with the retractor design, all or one of the bunker manifolds 53 may optionally use negative pressure. Thus, negative pressure alone, or with other bunker manifolds 53 delivering positive pressure, can be used.

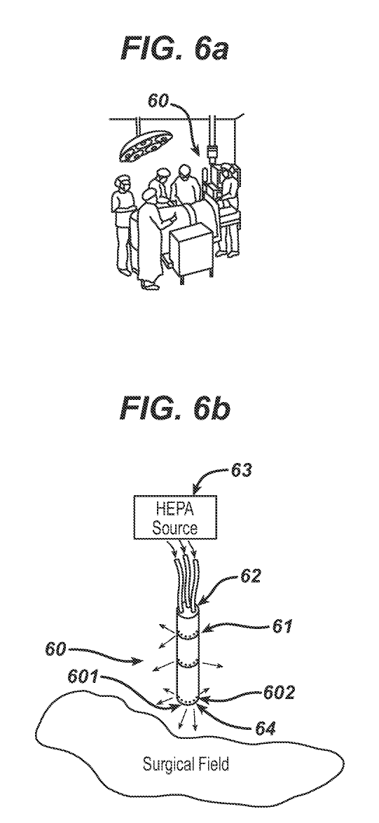

Another embodiment of the present invention is illustrated in FIG. 6a. A hollow cylinder 60 of a sufficiently effective length, for example approximately about 4-16 inches in length, is seen to be suspended from the ceiling 67 or a bracket attached to the operating table 68 or instrument stand 69. In one embodiment, the instrument stand 69 is a Mayo stand. As seen in FIG. 6b, the hollow cylinder 60 has a sufficiently effective cross-section, for example a diameter of approximately 1-3 inches and is essentially a tube with at least one row of holes 61 placed at a certain point along its length. The cylinder 60 has a proximal end 62 connected to at least one source of HEPA filtered air 63. For every row of holes 61, there is a dedicated source of HEPA filtered air. Holes 61 are in communication with the interior lumen or cavity 69 of cylinder 60. The distal end of the device 601 hangs above the surgical field at a height that does not interfere with surgeon's or assistants' hands or arms. The distal end of the device 601 may also be weighted with a weight 602 to ensure proper orientation of the cylinder, i.e., normal relative to the operating table plane. The bottom of the device 601 may also include a UV or blue light source 64 that can provide anti-microbial activity to the surgical site or surgical instruments. If a given airstream emitted by device 801 is interrupted, the other rows may still be intact, serving as backup airstreams.

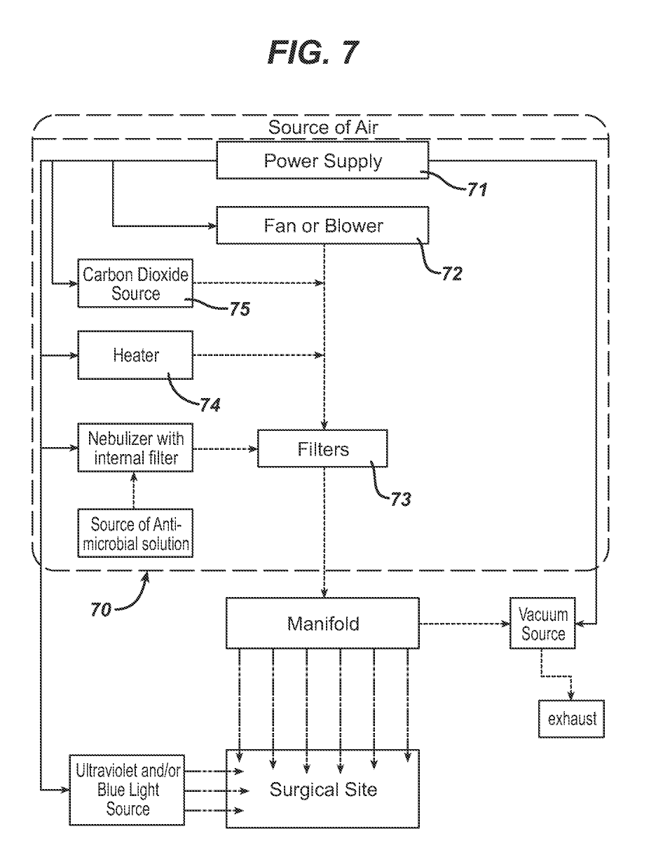

FIG. 7 illustrates a schematic of the system source of air 70 to any of the manifolds previously described herein. The source 70 is schematically illustrated within the dashed lines. The source 70 is generally comprised of a conventional power source 71 for a fan 72 or blower, the output of which passes through at least one filter 73. The source of air 70 may also be an existing laminar air flow system already built into the operating room structure, or a source of compressed or pressurized air. In one embodiment, there is a pre-filter that excludes particles of 5 microns or more, for example. The filtered air may optionally be passed through another filter that excludes bacteria and other microbes such as fungi and viruses. A filter with a porosity of 0.22-0.30 microns or less would be suitable for the second stage filter. Alternatively, one or more filters with 0.22-0.30 microns can be used. The air can be heated by any means known to those skilled in the art of heating air, such as a resistive element or heater 74 being near the air. In other embodiments, sterile solutions of anti-microbial or anti-biotic agents can be admixed with the air before or after filtration so that a germicidal effect is afforded to the surgical site. Suitable anti-microbial agents include antibiotics, triclosan, ethanol, or chlorhexidene gluconate in concentrations of 0.1-1.0 percent in a sterile saline or suitable physiological buffer such as phosphate buffered saline. Alternatively, the air can be pre-mixed with carbon dioxide by coupling a CO₂ generator 76 to the source. In addition, nebulized mists of anti-microbial solutions to further retard bacterial survival can also be utilized.

The present invention provides a great deal of freedom to the surgical staff with respect to exactly where and how each manifold delivers air. For example, air entering the manifold can optionally be pre-filtered with a HEPA filter, for example. In one embodiment, the air entering the manifold can be heated. Heating the air helps to avoid hypothermia, a condition known to increase the risk of surgical site infections. In another embodiment, the air is mixed with a dilute solution or mist of an antibiotic or anti-microbial agent. In another embodiment, the air is humidified with a sterile solution. The air may also be passed through a nebulizer just prior to entering the manifold. The HEPA filtered air can be directed in any number of directions and on multiple surgical sites, if these exist. In addition to adhesive as a way to attach the manifold to surfaces such as drapes, retractors, rails, clothing, etc., other conventional attachment devices or components such as velcro, pins, staples, tape, snaps, buttons, or sutures and the like can also be used.

Furthermore, the systems of the present invention can optionally be fitted with a source of UV (200-400 nm) or blue light (440-490 nm) to shine on the surgical site. The light can be placed on or attached to the manifold. The preferred wavelength of ultraviolet light is 254 nm. The preferred wavelength of blue light is 470 nm. Alternatively, the UV or blue light can be used separately, i.e., not attached to the manifold. Multiple manifolds can be used on one surgical site. At least one of these manifolds can be coupled to a negative pressure source coupled to a vacuum. The combination of a negative pressure source facing a positive air pressure may help to create a stronger and more aligned air flow to prevent intra-operative infection. For any of the embodiments illustrated herein, an “air curtain”, having a substantially non-laminar profile, can also be used. Although desired, laminar air flow per se is not a pre-requisite to the functioning of the device. In addition, while the drawings herein illustrate delivery heads with a plurality of holes, a single slit can also be used, the slit formed for at least a portion of the length of the air producing side of the manifold. The direction that the airflow is directed over the surgical site is easily adjusted by a surgeon or member of the surgeons staff by tilting or rotating any of the manifolds or shafts of the embodiments described herein. The preferred velocity of air flow for turbulent or laminar air flow profiles will be between about 25-300 feet per minute (FPM), and preferably within the range of about 70-120 FPM.

The following examples are illustrative of the principles and practice of the present invention, although not limited thereto.

### EXAMPLE 1

The system illustrated in FIG. 2 was tested for efficacy. The manifold was placed 6 inches above a petri dish containing 5% tryptic soy agar. The system was connected to an unfiltered source of air in a typical laboratory. A first person then rubbed their hands, wrists and forearms and neck over a petri dish at a height of approximately 18 inches for three minutes. A second person then rubbed their hands, wrists and forearms and neck over a different petri dish at a height of approximately 18 inches for three minutes. The attempted contamination of the dishes was referred to as the “inoculum”. In plate numbers 1-4, air was not supplied to the manifold. The number of colony forming units illustrates the utility of the system in reducing contamination. Bacteria found in the colonies was identified as Staphylococcus epidermidis and Staphylococcus aureus.

TABLE

| Plate | | Number of |
|---|---|---|
| Number | Conditions | colonies/plate |
| 1 | No air/No inoculum (negative control) | 2 |
| 2 | No air/No inoculum (negative control) | 0 |
| 3 | No air/Inoculum (positive control) | 112 |
| 4 | No air/Inoculum (positive control) | 4 |
| 5 | Air/No Inoculum (negative control) | 0 |
| 6 | Air/No Inoculum (negative control) | 0 |
| 7 | Air/Inoculum (single rod) | 19 |
| 8 | Air/Inoculum (single rod) | 5 |

Plate numbers 1, 3, 5, and 7 from first person.
Plate numbers 2, 4, 6, and 8 from second person.

### EXAMPLE 2

A surgical procedure that would benefit from the present invention is a surgical repair of a ventral hernia. The surgical team prepares for the surgical procedure in the following manner. A patient is prepared for surgery in a conventional manner by use of proper anesthesia, and prophylactic antibiotics, if necessary. The skin near and around the surgical site is cleaned with an antimicrobial agent such as iodine or chlorhexidine gluconate. The surgical site is then draped so as to minimize both the surgical site area and exposure of the surgical site to sites that were not cleaned by the surgical team. The system for reducing surgical site infection is placed near the proposed site of surgery. The flexible manifold illustrated in FIG. 4 is placed around the periphery of the surgical site. The device illustrated in FIG. 4 may also be placed around the periphery of the instrument stand. The devices contain a backing that is removable to expose a pressure sensitive adhesive. This adhesive allows the manifolds to be attached to the drapes covering the patient, a bed rail, or to the patient's skin, etc. The flexible manifolds are coupled to a HEPA air source by tubing that extends from the source and connects to the manifolds. Air is allowed to leave the holes in the manifold and travel over the desired height of the surgical site. The direction and height of the air flow are adjusted by the surgeon or the surgical team to meet the needs of the specific procedure. An incision is then made to initiate the surgery and the hernia is repaired. The surgeon and the surgical team keep the system in place until the wound has been closed and the dressings are applied. The system is turned off and the manifolds are removed from the surgical site and deposed of. The surgical team also has the option of using other components of the system illustrated in FIG. 7 during the procedure. For example, the use of blue light on the surgical site may also be utilized to further reduce the chance of a surgical site infection. In addition, the use of a nebulized mist of anti-microbial solution can also be employed to further reduce the chance of a surgical site infection. The nebulized mist can be applied directly to the surgical site or be directed to the manifold. The surgical team may also utilize carbon dioxide or heated air to reduce the likelihood of a surgical site infection.

Although this invention has been shown and described with respect to detailed embodiments thereof, it will be understood by those skilled in the art that various changes in form and detail thereof may be made without departing from the spirit and scope of the claimed invention.

1. A system for reducing infection during a surgical procedure, the system comprising: a source of air; a manifold comprised of a wall, a proximal end, distal end, and an interior lumen, wherein the lumen is coupled to the source of air by a conduit; at least one opening in the wall of the manifold in communication with the lumen; and, attachment means for attaching the manifold to a substrate proximate a surgical site, wherein said air additionally comprises an antimicrobial agent selected from the group consisting of chlorhexidene gluconate, triclosan, ethanol, and antibiotics and combinations thereof.

2. The system of claim 1, wherein said source of air is comprised of a power supply, a fan, a filter, and a coupling conduit for delivering the air to the manifold.

3. The system of claim 2, wherein the coupling conduit comprises tubing.

4. The system of claim 2, wherein the filter comprises a high efficiency particulate air filter.

5. The system of claim 3, wherein the tubing is comprises a material selected from the group consisting of a metal, plastic, and ceramic material.

6. The system of claim 1, wherein said air is under negative pressure.

7. (canceled)

8. The system of claim 1, wherein said attachment means is selected from the group consisting of a clamp, a shaft, bracket, screw, adhesive, cable, chain, wire, staple, and velcro.

9. The system of claim 8, wherein the attachment means comprises a shaft and wherein the shaft comprises a proximal end and a distal end; the proximal end of the shaft adapted to be mounted to the substrate, and the distal end of the shaft is attached to the manifold.

10. The system of claim 1 or 9, wherein the substrate is selected from the group consisting of surgical drapes, surgical retractors, beds, bedrails, or instrument stands.

11. The system of claim 9, wherein the shaft is flexible or articulated.

12. The system of claim 9, wherein the manifold is pivotably coupled to the distal end of the shaft.

13. The system of claim 1, further comprising an ultraviolet light producing a wavelength within the range of 200-400 nanometers, or a blue light source, producing a wavelength of 440-490 nm.

14. A method for performing a surgical procedure, the method comprising: providing a system comprising a source of air; a manifold comprised of a wall, a proximal end, a distal end, and an interior lumen, wherein the lumen is coupled to the source of air, the wall having at least one opening in communication with the lumen; attachment means for attaching the manifold to a substrate proximate a surgical site; and, directing the air from at least one opening in the manifold over a surgical site, wherein said air additionally comprises an antimicrobial agent selected from the group consisting of chlorhexidene gluconate, triclosan, ethanol, and antibiotics.

15. The method of claim 13, wherein said source of air comprises a power supply, a fan, a filter, and a coupling conduit for delivering the air to the manifold.

16. The method of claim 14, wherein the coupling conduit comprises of tubing.

17. (canceled)

18. The method of claim 13, wherein said attachment means is selected from the group consisting of a clamp, a shaft, bracket, screw, adhesive, cable, chain, wire, staple, and velcro.

19. The method of claim 18, wherein the attachment means comprises a shaft, the shaft comprising a proximal end and a distal end, wherein the proximal end of the shaft is adapted to be secured to a retractor, bed, bedrail, or instrument stand, and, the distal end of the shaft is attached to the manifold.

20. The method of claim 19, wherein the shaft is flexible or articulated.

21. The method of claim 13, wherein the system further comprises an ultraviolet light having a wavelength within the range of 200-400 nanometers or a blue light source with a wavelength of 440-490 nanometers.

22. The method of claim 16, wherein the air leaving the manifold is laminar flow air.

23. The method of claim 13, wherein the surgical site is a surgical wound, an instrument stand, or a hospital bed.

24. The system of claim 1, wherein the source of air comprises compressed air.

25. The system of claim 1, wherein the source of air comprises pressurized air.

26. The method of claim 14, wherein the source of air comprises compressed air.

27. The method of claim 14, wherein the source of air comprises pressurized air.
